(19) United States
(12) Patent Application Publication
(10) Pub. No.: US 2009/0315348 A1
(43) Pub. Date: Dec. 24, 2009

(54) System and method for use of a releasable access tool by disabled persons

(76) Inventor: Stephen Paul Diebold, Rolling Meadows, IL (US)

Correspondence Address: Stephen Paul Diebold, 2702 Mill Creek Lane, Rolling Meadows, IL, 60008, US

(21) Appl. No.: 12/215,024

(22) Filed: Jun. 24, 2008

(51) Int. Cl. A61F 4/00; B25J 11/00

(52) U.S. Cl. 294/1.1

(57) ABSTRACT

A releasable access tool and method for use by disabled persons is provided which includes a chin-mounted pointer that can be engaged and disengaged by a user without assistance. The releasable access tool includes a chin cup and chin cushion, a means for suspending them from the neck of a disabled person, and a pointer affixed to the chin cup. Assistance is required in the placement of the releasable access tool around the neck of a user. Once the device is placed around the user's neck, the user mounts the chin cup and pointer to the user's chin by lowering the chin to contact the cup, then lifting the chin and head in a scooping motion. This stretches the neck suspension portion of the tool and pulls the chin cup firmly against the user's chin, allowing the user to manipulate object with the attached pointer using only the muscles of the face, head and neck. When the user wishes to stop using the pointer, the user lowers the chin and presses the chin toward the chest and neck to allow the chin cup to slip from the chin. The releasable access tool then hangs in a neutral position around the neck of the user until it is accessed again by the user or until it is removed from the user's neck by an assistant.

### CROSS-REFERENCE TO RELATED APPLICATIONS

Not Applicable

### BACKGROUND OF THE INVENTION

The present invention generally relates to a releasable access tool for use by disabled persons. More particularly, the present invention relates to a releasable access tool that is used by a person living with a disability to physically move or manipulate a target object.

Modern electronic communications devices such as the personal computer have vastly improved public access to sources of information and entertainment. This access is especially important to persons living with disabilities that adversely impact mobility and manual dexterity. However, the designs of these devices require user input through typing, pressing buttons, operating toggle switches or track wheels, and other forms of physical interaction that may be difficult or impossible for those who have limited use of hands or arms. Quadriplegia, in particular, presents challenges in the use of devices and objects that are designed to be manipulated by hand. Persons with quadriplegia may have volitional control over the muscles of the head and neck only. This limits the usefulness of the personal computer for persons with quadriplegia because they are unable to type or to operate a standard mouse.

In addition to the difficulties encountered in the use of keyboards and various electronic devices, persons without the use of the hands or arms face a wide variety of environmental obstacles. Light switches, elevator buttons and other objects that must be touched or pushed severely limits the ability of the person with a disability to perform tasks both at work and in the home. Using the muscles of the head and neck to replace in some part the lost function of the hands and arms greatly increases the independence and professional functionality of a person living with a disability. Several devices have been designed to assist in the physical manipulation of objects (such as the keys of computer keyboards) through the use of a pointer that is controlled by one or more muscles of the neck, mouth and head.

One such access device is disclosed in Haley, U.S. Pat. No. 5,422,640. The device described by Haley allows a paralyzed user to use a computer keyboard. A keyboard is mounted on a movable tilted base to which a vertical support arm is attached. The base holds the keyboard at an adjustable angle. A pointer is mounted to the opposite end of the support arm and pivots on the arm; the proximal end of the pointer is controlled by the face and mouth of the user, while the distal end of the pointer engages the keyboard. Head movements of the user move the pointer horizontally across the keyboard. Vertical movement of the pointer is produced when the user blows a breath into the pointer's breath port. The breath is detected by a pressure sensor that actuates a solenoid in response, triggering the extension of the distal end of the pointer and causing it to strike the selected key.

While this device increases the accessibility of computers for disabled persons, it requires a relatively large apparatus that is visually apparent to others. In addition, the device is limited in function to the operation of a keyboard. The user's ability to speak, eat and drink is limited while the device is in use. This device requires an assistant to adjust the angle of the keyboard and pointer for the user, and also may require periodic maintenance.

A second access tool is disclosed in Sauer et al., U.S. Pat. No. 4,828,418. This device is a mouth-held pointer comprising a wedge-shaped plastic portion to which a pointer shaft is attached. The plastic wedge, which is held in the user's mouth, is fitted to a user's own mouth by heating the wedge to soften the plastic and placing the wedge in the mouth of the user to form an impression of the user's teeth and other surrounding anatomical structures. When cooled, the plastic retains the contours of the mouth, allowing the device to fit more comfortably. The user then holds the molded plastic wedge in the mouth with the pointer shaft projecting forward. The user utilizes the musculature of the head and neck to physically manipulate objects with the distal end of the pointer.

The mouth-held pointer is a relatively economical device that can be used for a variety of tasks. However, there are several drawbacks to using mouth-held pointers. This kind of pointer requires the help of an assistant, who must place the pointer in the user's mouth. Holding the pointer in the mouth causes some users to salivate, causing embarrassment and discomfort. Mouth-held pointers may cause fatigue of the jaw muscles and possible dental erosion. In addition, the user cannot speak, eat or drink while using the pointer and the ability to breathe through the mouth is also compromised during use.

A design for a head-mounted access tool is disclosed in Johnson, U.S. Pat. No. D312,169. This device is comprised of a circular band placed horizontally around the upper circumference of the user's head, a curved support attached to one side of the band and extending upward, and a pointer coupled to the curved support. The proximal end of the pointer is positioned above the user's head and extends forward and down, placing the pointer in front of the user's face. This allows the user to physically manipulate objects with the distal end of the pointer by moving the muscles of the head and neck.

One drawback of this device is that it is visually very noticeable, causing social discomfort to the user. An assistant must place the device on the head of the user before the user can operate it, and an assistant's help is also required to remove the device when the user is finished. Because the pointer extends downward over the face of the user, the user's field of vision is partially obscured while the device is in use, causing discomfort and strain on the eyes, particularly where the user is operating a keyboard with the pointer while reading a computer monitor positioned behind the pointer.

Other head pointers commercially available or described in the prior art include pointers mounted to helmets or other headgear worn on the user's head, pointers strapped to the user's forehead, pointers held in the user's mouth, and pointers held to the user's chin with straps or other connectors that are wrapped around the user's head above the ears. Each of these pointers commonly requires a person other than the user to secure the pointer to the user's head or within the user's mouth each time the device is to be used. Once secured, the pointer is utilized by the user to manipulate objects using the muscles of the user's head, neck, face and/or mouth. When the user has completed the desired tasks, a person other than the user must remove the pointer from the user's head and/or mouth until the next time the pointer is used. The devices are visually noticeable, causing social discomfort to the user. The cost of the pointers is variable, with devices ranging from less than $100 for basic models to well over $1000 in price for pointers that incorporate electronic components for interaction with computer software. Cost is a serious issue for those with disabilities and living on fixed incomes, but also for employers, who are required under the ADA to make reasonable accommodations for employees with disabilities.

### BRIEF SUMMARY OF THE INVENTION

One or more of the embodiments of the present invention provide a method and apparatus for the physical manipulation of objects by a disabled person using the muscles of the head and neck. Furthermore, one or more of these embodiments allows the user to engage in using the device and to disengage from the device without the assistance of others. Embodiments of the present invention describe a chin-mounted pointer that is worn around the neck of the user. These embodiments allow the user to engage the muscles of the head and neck to pick up the pointer, manipulate a target object with the pointer, and subsequently drop the pointer from the chin using the muscles of the head and neck when the user has completed a task. The pointer is then available for further engagement by the user, without the assistance of others, when the user wishes to perform another task with the pointer.

### BRIEF DESCRIPTION OF THE DRAWINGS

FIG. 1 illustrates a perspective view of a releasable access tool according to an embodiment of the present invention.

FIG. 2 illustrates a plan view of a variable shaft position chin cup according to an embodiment of the present invention.

FIG. 3 illustrates a perspective view of a releasable access tool featuring a continuous neck strap according to an embodiment of the present invention.

FIG. 4 illustrates a perspective view of a releasable access tool featuring a spring-loaded neck retention element according to an embodiment of the present invention.

FIG. 5a illustrates a first step in the use of a releasable access tool according to an embodiment of the present invention, wherein the releasable access tool is suspended in a neutral position around the neck of the user prior to use.

FIG. 5b illustrates a second step in the use of a releasable access tool according to an embodiment of the present invention, wherein a user's head and neck are positioned to engage the releasable access tool.

FIG. 5c illustrates a third step in the use of a releasable access tool according to an embodiment of the present invention, wherein a user's head, chin and neck are positioned for use of the releasable access tool mounted upon the user's chin.

FIG. 6 illustrates a flow chart for a method of use of a releasable access tool according to an embodiment of the present invention.

### DETAILED DESCRIPTION OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The terms “coupled” and “connected,” along with their derivatives, may be used. It should be understood that these terms are not interchangeable. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

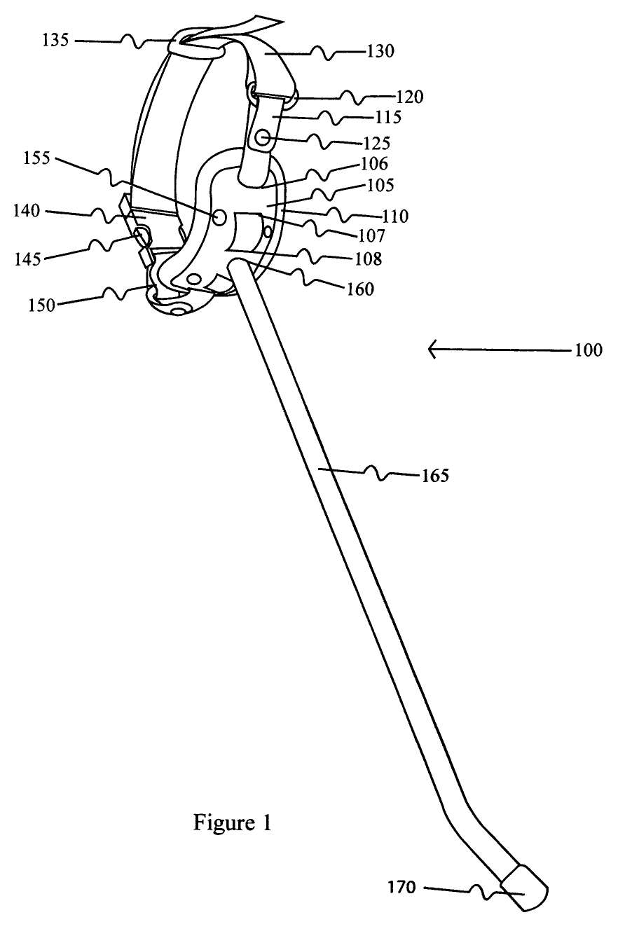

FIG. 1 illustrates a perspective view of a releasable access tool 100 for use by a disabled person according to an embodiment of the present invention. The embodiment of an access tool 100 includes a chin cup 105 with an interior concavity shaped to accommodate a chin, a chin cushion 110, a front strap 115, connector rings 120, front strap snaps 125, a neck strap 130, a neck strap length adjustor 135, a side release buckle outer member 140, a side release buckle inner member 145, a side release buckle strap 150, ventilation holes 155, a shaft seat 160, a shaft 165, and a shaft tip 170. The chin cup 105 further includes side apertures 106, outer front apertures 107 and inner front apertures 108.

In the preferred embodiment, the chin cup 105 is coupled along its inner surface with the chin cushion 110. Four ventilation holes 155 pass from the outer surface of the chin cup 105 through the chin cushion 110. The front strap 115 is mechanically affixed to the chin cup 105 and to the chin cushion 110 by passing the front strap 115 through the side apertures 106, the outer front apertures 107 and the inner front apertures 108, such that the front strap 115 protrudes from the outer surface of the chin cup 105 through the side apertures 106. The front strap 115 is positioned between and in contact with the chin cup 105 and the chin cushion 110 between the side apertures 106 and the outer front apertures 107, and again between the inner front apertures 108. The front strap 115 remains on the outer surface of the chin cup 105 between the outer front apertures 107 and the inner front apertures 108. Each end of the front strap 115 is passed through a connector ring 120 and is secured to the connector ring 120 by a front strap snap 125.

On one side of the releasable access tool 100, a connector ring 120 is coupled to one end of the neck strap 130. The free end of the neck strap 130 is passed through the interior of the neck strap length adjustor 135. The neck strap 130 further extends to the side release buckle outer member 140. The neck strap 130 is passed through the side release buckle outer member 140 and the free end of the neck strap 130 is passed again through the neck strap length adjustor 135.

On the opposite side of the releasable access tool 100, a connector ring 120 is coupled to the side release buckle strap 150. The side release buckle strap 150 is coupled to the side release buckle inner member 145. The side release buckle inner member 145 is reversibly coupled to the side release buckle outer member 140.

The shaft seat 160 is embedded in the chin cup 105. The proximal end of the shaft 165 is coupled to the shaft seat 160, with the shaft 165 protruding from the outer surface of the chin cup 105. The distal end of the shaft 165 is coupled to the shaft tip 170.

In operation, a person other than the user (an assistant) prepares the releasable access tool 100 for use by uncoupling the side release buckle inner member 145 from the side release buckle outer member 140. The assistant places the releasable access tool 100 around the neck of a disabled person. Next, the assistant couples the side release buckle outer member 140 to the side release buckle inner member 145, securing the releasable access tool 100 around the neck of the user. The assistant adjusts the length of the neck strap 130 by pulling the free end of the neck strap 130 through the neck length strap adjustor 135 to shorten the neck strap 130, or by pulling some portion of the neck strap 130 in the opposite direction through the neck strap adjustor 135 to lengthen the neck strap 130. The assistant then releases the neck strap 130, allowing the releasable access tool 100 to rest around the lower neck and upper chest of the disabled person, with the chin cup 105 and the chin cushion 110 resting below the disabled person's chin.

The disabled person (the user) employs the muscles of the head and neck to push their chin toward their lower neck, placing the front surface of their chin in physical contact with the chin cushion 110. The user then employs the muscles of the head and neck to push their chin outward and upward from their lower neck with the chin cushion 110 in physical contact with the front surface of the their chin. This alteration of the user's head and chin position places tension on the neck strap 130, which pulls the chin cup 105 and the chin cushion 110 toward the user and seats the chin cushion 110 and the chin cup 105 securely on the user's chin. The user then moves the shaft 165 by moving their chin, head or neck until the shaft tip 170 is in physical contact with a target object. The user subsequently applies force against the target object by moving the their chin, head or neck, allowing the user to manipulate the target object.

The user disengages the releasable access tool 100 from their chin by using the muscles of the neck, head or chin to lower their chin toward their lower neck. This movement reduces the pull placed on the chin cup 105 and the chin cushion 110 by the neck strap 130 until the force of gravity is greater than the functional tension generated by the neck strap 130, allowing chin cup 105 and chin cushion 110 to drop from the user's chin onto the user's lower neck or chest. The user leaves the releasable access tool 100 around the user's lower neck until it is either accessed again by the user or removed by the assistant. At some later time, the assistant releases and removes the releasable access tool 100 from the user's lower neck by uncoupling the side release buckle outer member 140 from the side release buckle inner member 145.

In the preferred embodiment, the chin cup 105 is constructed of translucent, colorless plastic. Some embodiments include a chin cup 105 constructed of acrylic, while other embodiments include a chin cup 105 constructed styrene. The chin cup 105 may also be constructed of another clear rigid or semi-rigid material including, but not limited to, polycarbonate, polyvinylchloride, polyurethane, or polypropylene. In some embodiments the chin cup 105 includes an elastomer. In an embodiment, the chin cup 105 is constructed of an opaque material and is flesh-colored. In some embodiments, the chin cup 105 is constructed of a translucent colored material while in other embodiments the chin cup 105 is constructed of an opaque colored material. In an alternative embodiment, the chin cup 105 is adapted to extend along the length of the user's jaw. The chin cup 105 covers only the user's chin in other embodiments. The chin cup 105 is formed in various embodiments to accommodate different chin sizes and shapes.

In the preferred embodiment, the chin cushion 110 is adhesively affixed to the surface of the interior concavity of the chin cup 105 and to a narrow margin along the outer circumference of the exterior surface of the chin cup 105. In some embodiments the chin cushion 110 covers only the surface of the interior concavity of the chin cup 105. Some embodiments are constructed without a chin cushion 110. The chin cushion 110 is elastic and can be secured to the chin cup 105 by stretching the chin cushion 110 over the chin cup 105 in some embodiments. In an embodiment, the interior concavity of the chin cup 105 is coated with a compressible material in place of the chin cushion 110. In another embodiment, the chin cushion 110 is a flexible hollow cylindrical component open at both ends that is pulled over the chin cup 105 to cover both the interior concavity and the exterior surface of the chin cup 105. Some embodiments further include a compressible pad positioned between the concavity of the chin cup 105 and the chin cushion 110. This compressible pad may be shaped and sized to accommodate the chin of the user in some embodiments.

In the preferred embodiment, the chin cushion 110 is made of silicone. In other embodiments, other translucent and colorless compressible materials are used to make the chin cushion 110. The chin cushion 110 is solid in some embodiments, while in other embodiments the chin cushion 110 is hollow and filled with a fluid such as air, water, or gel. In an embodiment, the chin cushion 110 is opaque. In some embodiments, the chin cushion 110 is flesh-colored. In another embodiment, the chin cushion 110 is both opaque and colored. In an embodiment, the chin cushion 110 is removably affixed to the chin cup 105 using snaps, Velcro, suction, a dissolving adhesive or another fastener.

The chin cup 105 and the chin cushion 110 include four round ventilation holes 155 in the preferred embodiment. Some embodiments include fewer than four ventilation holes 155, while other embodiments include more than four ventilation holes 155. Several embodiments are constructed without the ventilation holes 155. In an embodiment, vertical slits pass through the chin cup 105 for ventilation. In other embodiments, the chin cup 105 includes ventilation apertures of varying shapes and sizes. In some embodiments, the chin cup 105 includes one, two, three, four, five, six or more ventilation apertures.

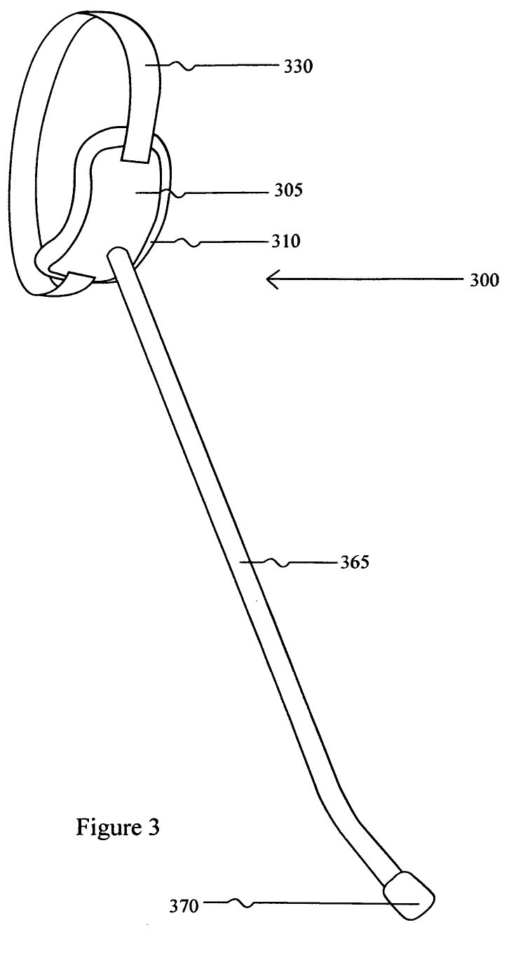

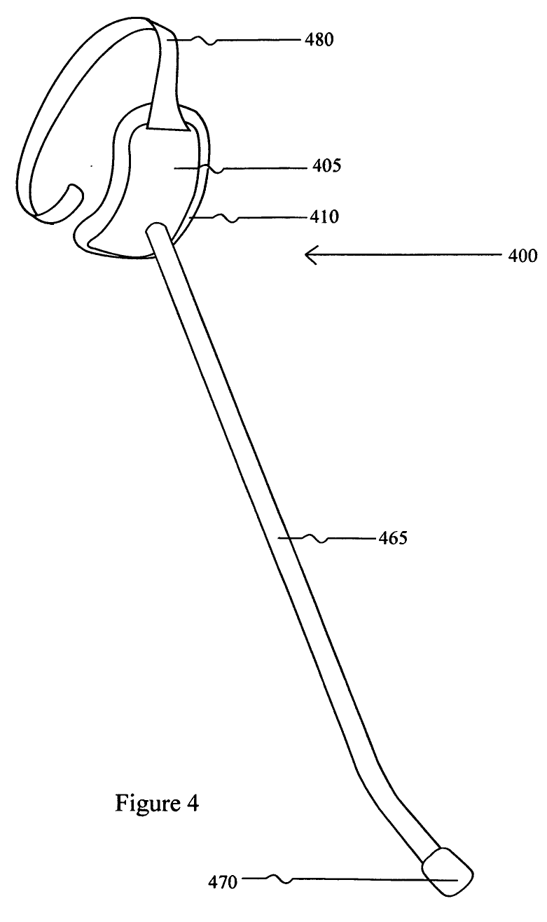

In the preferred embodiment, the front strap 115 is in physical contact with a portion of the outer surface area of the chin cup 105 and with a portion of the surface of the chin cushion 110. In other embodiments, the front strap 115 is adhesively affixed to the outer surface of the chin cup 105. In an embodiment, the front strap 115 is adhesively affixed to one front surface portion of the chin cup 105 and another front strap 115 is adhesively affixed to another front surface portion of the chin cup 105. Some embodiments are constructed without the side apertures 106, while other embodiments are constructed without the outer front apertures 107 and the inner front apertures 108. In an embodiment, the chin cup 105 and the front strap 115 are formed as a single unit. Some embodiments are constructed without the front strap 115 (see FIG. 3 and FIG. 4). In an embodiment, the front strap 115 is composed of silicone. In other embodiments, the front strap 115 includes leather, plastic, elastic, rubber, metal, or another material.

In the preferred embodiment, the front strap 115 is passed through a connector ring 120 and is secured to itself by a front strap snap 125 to form a loop. Some embodiments are constructed without a front strap snap 125, while other embodiments are constructed without a connector ring 120 (see FIG. 3 and FIG. 4). The front strap snap 125 is metallic in the preferred embodiment. In another embodiment the front strap snap 125 is plastic. In some embodiments, a rivet replaces the front strap snap 125. In other embodiments the front strap 115 is adhesively affixed to a connector ring 120. In an embodiment the front strap 115 is passed through a connector ring 120 and is adhesively affixed to itself to form a loop. In an embodiment, the front strap 115 and a connector ring 120 are formed as a single component, while in other embodiments the front strap 115 is removably coupled to a connector ring 120. In some embodiments a connector ring 120 is plastic. In other embodiments a connector ring 120 is metal. The shape of the connector ring 120 varies among embodiments.

The preferred embodiment includes a neck strap 130 and a side release buckle strap 150. In the preferred embodiment, both the neck strap 130 and the side release buckle strap 150 include an elastic component that allows stretching of the straps in response to applied force. In some embodiments, the side release buckle strap 150 does not include an elastic component. Other embodiments are constructed without a side release buckle strap 150 (see FIG. 3 and FIG. 4). While the neck strap 130 and the side release buckle strap 150 are each coupled to a connector ring 120 in the preferred embodiment, in some embodiments both the neck strap 130 and the side release buckle strap 150 are coupled directly to the chin cup 105.

In an embodiment, the neck strap 130 is doubled over to allow resizing around the neck of the user. Some embodiments do not include a means of resizing the neck strap 130. In an embodiment, the neck strap 130 is shortened to accommodate a child or adult of small size. Some embodiments include two, three or four neck straps 130 to redistribute the force applied to the neck during use of the releasable access tool 100. In the preferred embodiment, the neck strap 130 is elastic along its entire length. In other embodiments the neck strap 130 is not elastic, and another component such as the front strap 115 is elastic. In an embodiment the neck strap 130 is flesh-colored, while in another embodiment the neck strap 130 is colorless. The neck strap 130 is made of a nylon-spandex blend in the preferred embodiment; other embodiments include a neck strap 130 made at least partially of silicone, plastic, leather, rubber, or other materials. In an embodiment, the portion of the surface of the neck strap 130 that is placed in contact with the neck of the user during use of the releasable access tool 100 is coated in one or more places with an elastomer to prevent slippage of the neck strap 130 along the neck of the user.

In some embodiments, the neck strap 130 includes a slidably adjustable neck cushion. In other embodiments, the neck strap 130 includes a permanently affixed neck cusion. In an embodiment, a slidably adjustable neck cushion comprises a compressible pad with a snap, Velcro, or other fastener; in this embodiment, the slidably adjustable neck cushion is wrapped around the neck strap 130 and is fastened to itself. In another embodiment, a slidably adjustable neck cushion comprises a compressible pad with one or more slits at each of two ends, connecting to an interior lumen through which the neck strap 130 is threaded. In an embodiment, the neck strap 130 does not include an interior lumen and has one or more loops coupled to the exterior of the neck cushion through which neck strap 130 is threaded. In some embodiments, the neck cushion is not slidably adjustable along the neck strap 130, does not have an interior lumen and is sewn, glued, snapped, buckled, affixed with Velcro, or otherwise affixed to the neck strap 130.

The preferred embodiment includes a side release buckle inner member 145 and a side release buckle outer member 140, which are removably coupled to fasten releasable access tool 100 around the neck of a user. Other embodiments are constructed without a side release buckle inner member 145 and a side release buckle outer member 140 (see FIG. 3 and FIG. 4). In some embodiments, the side release buckle inner member 145 and the side release buckle outer member 140 are replaced with a snap. In an embodiment, Velcro is used to removably couple one end of the neck strap 130 to another portion of the neck strap 130. In another embodiment, Velcro is used to removably couple the neck strap 130 to the chin cup 105. Some embodiments include a plastic side release buckle while others include a metal side release buckle, a plastic or metal front release buckle, or another variety of buckle.

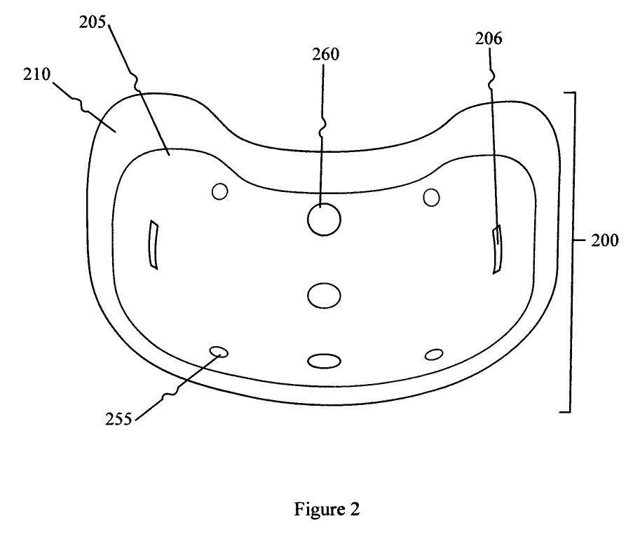

In the preferred embodiment, a single shaft seat 160 is located at the center of the chin cup 105. In an alternate embodiment, the chin cup 105 includes multiple openings that can accommodate the shaft 165, such that the shaft 165 can be placed in one of multiple positions on the chin cup 105 (see FIG. 2). In the preferred embodiment, the shaft 165 is a translucent, colorless acrylic rod with a hollow interior and a 15 degree bend approximately three inches from the distal end. In another embodiment, the shaft 165 is opaque. In some embodiments, the shaft 165 is colored. In other embodiments, the shaft 165 is both opaque and colored. In an embodiment, the shaft 165 is removably coupled to the chin cup 105. In some embodiments, the shaft 165 is threaded at one end and is removably coupled to the chin cup 105 by inserting the threaded end into an aperture in the chin cup 105 or into a shaft seat 160 and turning one of more of those elements. In other embodiments, the shaft 165 is permanently affixed to the chin cup 105. In an embodiment, the shaft 165 does not have a hollow interior and is solid. In some embodiments, the shaft 165 is a translucent, colorless rod that is constructed of a non-acrylic material, such as glass, polycarbonate, polyvinyl chloride, polypropylene, polystyrene, or epoxy. In other embodiments, the shaft 165 is constructed of a colored or non-translucent material such as a metal or a metal alloy, plastic, or ceramic. In an embodiment, the shaft 165 is constructed without a bend along the distal portion. In another embodiment, the shaft 165 includes a bend along the proximal portion while the distal portion is straight. In some embodiments, the bend in the shaft 165 is greater than 15 degrees, while in other embodiments, the bend in the shaft 165 is less than 15 degrees. In an embodiment, the shaft 165 is constructed with a hollow interior that encloses an electronic module adapted for interaction with other electronic devices. In another embodiment, the shaft 165 is constructed with a hollow interior that encloses a camera. In one embodiment, the shaft 165 is constructed with a hollow interior that accommodates a laser pointing device, while in another embodiment the shaft 165 is constructed with a hollow interior that accommodates a light source.

In the preferred embodiment, the shaft tip 170 is constructed of clear silicone. Some embodiments are constructed without a shaft tip 170. In an embodiment, the shaft tip 170 is constructed of colored silicone. In some embodiments, the shaft tip 170 is constructed of a material other than silicone, such as rubber or plastic. In other embodiments, the shaft tip 170 has a textured surface that increases its surface area. In embodiments, the shaft tip 170 is removably coupled to the shaft 165. In some embodiments, the bottom surface of the shaft tip 170 incorporates a functional component such as a pen, a pencil, a piece of chalk, a paint brush, a magnet, Velcro, a laser pointer, a light source, a camera, a hook, or a suction cup. In an embodiment, the shaft tip 170 is reversible and incorporates a functional component at each end, such that one component projects from the distal portion of the shaft tip 170 during use while the other is retained within the interior of a hollow shaft 165. In this embodiment, the user has the option of removing the shaft tip 170 and reattaching the shaft tip 170 to a hollow shaft 165 such that the positions of the components of the shaft tip 170 relative to the shaft 165 are reversed (allowing the user to use the component formerly retained within the shaft 165). For example, in an embodiment, the shaft tip 170 includes a paintbrush component and a chalk component; while one projects from the distal end of the shaft tip 170, the other is retained within the shaft 165.

In some embodiments, the shaft tip 170 is coupled to the shaft 165 by magnetic elements included in both the shaft tip 170 and the shaft 165, such that the shaft tip 170 is removable by a user while the releasable access tool 100 is in use. In an embodiment, one or more variations of the shaft tip 170 are arranged in a container adapted to releasably retain various embodiments of the shaft tip 170 in rigidly fixed positions with their magnetic elements accessible. In this embodiment, a user changes from a first shaft tip 170 to a second shaft tip 170 during use of a releasable access tool by using the muscles of the face and neck. The user places the shaft tip 170 that is attached to the releasable access tool into contact with the portion of the container adapted to retain a shaft tip 170, applying downward force to the shaft tip 170 until the shaft tip 170 is securely retained within the container. The user then removes the shaft tip 170 by using the muscles of the face or head to apply downward force at an angle to the shaft tip 170 through the shaft 165, overcoming the magnetic force and uncoupling the shaft tip 170 from the shaft 165. The user subsequently selects a different shaft tip 170 and attaches that shaft tip 170 by placing the distal end of the shaft 165 against the exposed magnetic element of the selected shaft tip 170. The user lifts the selected shaft tip 170, now magnetically coupled to the shaft 165, from the container by applying upward force to the shaft tip 170 through the shaft 165.

In another embodiment, a variety of the shaft tip 170 embodiments are arranged in a container adapted to releasably retain various embodiments of the shaft tip 170 in flexibly fixed positions with their magnetic elements accessible. In this embodiment, a user replaces a first shaft tip 170 with a second shaft tip 170 during use of a releasable access tool by using the muscles of the face and neck. The user places the shaft tip 170 that is attached to the releasable access tool into contact with the portion of the container adapted to retain a shaft tip 170, applying downward force to the shaft tip 170 until the shaft tip 170 is securely retained within the container. The user then removes the shaft tip 170 by using the muscles of the face or head to apply upward force to the shaft tip 170 through the shaft 165, overcoming the magnetic force and uncoupling the shaft tip 170 from the shaft 165. The user subsequently selects a different shaft tip 170 and attaches that shaft tip 170 by placing the distal end of the shaft 165 against the exposed magnetic element of the selected shaft tip 170. The user then applies downward force at an angle to the shaft tip 170 through the shaft 165, releasing the shaft tip 170 from the flexible portion of the container in which it was retained.

In alternative embodiments, the shaft tip 170 is removably coupled to the shaft 165 by other means. In an embodiment, the shaft tip 170 and the shaft 165 are coupled using a snap mechanism, such as a long prong snap, a gripper snap, or a short prong snap. In another embodiment, the shaft tip 170 has elastic properties and is removably coupled to the shaft 165 by pushing the distal end of the shaft 165 into an interior concavity of the shaft tip 170.

In some embodiments, the shaft tip 170 has two or more surfaces of different textures; in one embodiment the bottom of the shaft tip 170 is covered with small elastic projections while the sides of the tip shaft 170 are smooth. In an embodiment, the shaft tip 170 is adapted for turning the pages of a book.

The shaft tip 170 is adapted in some embodiments to allow the user to grasp objects. In some of these embodiments, the shaft 165 is hollow and accommodates within its interior some portion of a grasping device; a claw end comprising one, two, three or more opposed prongs protrudes from the end of the shaft 165 distal to the user while a pressure-sensitive element coupled to the grasping device projects from the chin cup 105 at the end of the shaft 165 proximal to the user. The pressure-sensitive element is held within the mouth or between the lips of the user. Applying pressure to the pressure-sensitive element with the lips, tongue, mouth or teeth causes the prongs of the claw to move away from one another at the ends of the prongs most distal to the user, creating an interior space within the claw end in which to grasp an object. The user manipulates the open claw end into position around a target object and subsequently releases the pressure applied to the pressure-sensitive element, causing the prongs of the claw end to move toward one another at the ends of the prongs most distal to the user. The closing of the claw end around the target object allows the user to move or manipulate the target object. To release the target object, the user applies pressure to the pressure-sensitive element, causing the prongs of the claw end to move apart.

In some embodiments, the grasping device is incorporated into the releasable access tool, while in other embodiments the grasping device is added or removed by an assistant. In some embodiments, the grasping device is activated by a pressure-sensitive solenoid that responds to air pressure generated by the user. In an embodiment, applying pressure to a pressure-sensitive element causes the prongs of the claw end to close; in other embodiments, applying pressure to a pressure-sensitive element causes the prongs of the claw end to open. In an embodiment, the claw end is retractable into the shaft 165 by the user.

FIG. 2 illustrates a plan view of a variable shaft position chin cup 200 according to an embodiment of the present invention. This embodiment of a variable shaft position chin cup 200 includes a chin cup 205, side front apertures 206, a chin cushion 210, ventilation holes 255, and three shaft apertures 260.

In the illustrated embodiment, the chin cup 205 is affixed along its inner surface and along the outer circumference of its outer surface to the chin cushion 210. Four ventilation holes 255 pass from the outer surface of the chin cup 205 through the chin cushion 210. Two side front apertures 206 pass through the chin cup 205 but do not pass through the chin cushion 210. Three shaft apertures 260 pass through the chin cup 205 but do not pass through the chin cushion 210.

In some embodiments, the chin cup 205 includes one shaft aperture, while in other embodiments, the chin cup 205 includes two, three, four or more than four shaft apertures. In an embodiment the interior surface of a shaft aperture 260 is threaded such that a shaft with a threaded proximal end may be affixed to the chin cup 205 by turning the threaded proximal end of the shaft in the shaft aperture 260. In another embodiment the interior surface of the shaft aperture 260 is smooth. In an embodiment, two, three, four or more than four shaft apertures 260 are arranged horizontally across the chin cup 205. In other embodiments, two, three, four or more than four shaft apertures 260 are arranged vertically across the chin cup 205. In an embodiment, two, three, four or more shaft apertures 260 are arranged equidistant from the vertical and horizontal center of the front surface of the chin cup 205. In another embodiment, two, three, four or more shaft apertures 260 are arranged in an irregular pattern across the chin cup 205. In an embodiment, the chin cup 205 includes a shaft aperture 260 that is placed above, below, to the left or to the right of the horizontal and vertical center of the chin cup 205. Some embodiments are constructed without a front side aperture 206, while others include two, four or more front side apertures 206. Although the illustrated embodiment includes four vent holes 255, some embodiments include one, two or three vent holes 255 while others include more than four vent holes 255. The concavity of the inner surface of the chin cup 205 varies among embodiments to accommodate different chin sizes.

FIG. 3 illustrates a perspective view of a releasable access tool 300 featuring a continuous neck strap according to an embodiment of the present invention. The illustrated embodiment includes a chin cup 305, a chin cushion 310, a neck strap 330, a shaft 365, and a shaft tip 370.

In the illustrated embodiment of a releasable access tool 300, the chin cup 305 is affixed along its inner surface and along the outer circumference of its outer surface to the chin cushion 310. The neck strap 330 is adhesively affixed to two sides of the chin cup 305. The proximal end of the shaft 365 is adhesively affixed to the front surface of the chin cup 305, and the shaft tip 370 is mechanically coupled to the distal end of the shaft 365.

In operation, the releasable access tool 300 is placed around the neck of a disabled person (the user) by another person (an assistant) by stretching the neck strap 330 to create a circumference greater than the circumference of the user's head at its widest point. The assistant then places the tool around the head of the user and pulls the tool over the head and down to the neck, with the chin cup 305 resting below the chin of the user. To access the tool, the user lowers the chin to contact the inner concavity of the chin cup 305 and subsequently pushes the chin forward to seat the chin cup 305 and the chin cushion 310 on the front surface of the user's chin. The user then lifts the user's head using the muscles of the neck and head. The neck strap 330 maintains a backward pull on the chin cup 305, keeping the chin cup 305 and the chin cushion 310 affixed to the user's chin. The user moves the muscles of the face, head and neck to move the shaft 365 or the shaft tip 370 into physical contact with a target object. The user then manipulates the target object by applying force against the shaft 365. To disengage the releasable access tool 300, the user lowers the chin and presses the chin toward the neck and chest, allowing the releasable access tool 300 to slip from the chin and rest in a neutral position around the neck of the user. An assistant removes the tool by stretching the neck strap 330 to create a circumference greater than the circumference of the user's head at the widest point of the user's head, subsequently pulling the tool over the head of the user.

The illustrated embodiment includes five separate components, but other embodiments include fewer separate components; for example, in one embodiment the chin cup 305 and the chin cushion 310 are constructed as a single piece. In another embodiment, the chin cup 305 and the shaft 365 are constructed as a single piece. In other embodiments, the shaft 365 and the shaft tip 370 are constructed as a single piece. In an embodiment, the chin cup 305, the chin cushion 310 and the neck strap 330 are constructed as a single piece. In some embodiments, the chin cup 305, the chin cushion 310, and the shaft 365 are constructed as a single piece. In another embodiment, the chin cup 305, the chin cushion 310, the neck strap 330 and the shaft 365 are constructed as a single piece. Some embodiments include multiple apertures adapted to simultaneously accommodate two, three or more shafts 365. Other embodiments include a mechanism such as Velcro, one or more snaps, or an adjustable buckle that is used to shorten and lengthen the neck strap 330. In the illustrated embodiment, the ends of the neck strap 330 are adhesively affixed to the chin cup 305 on two sides of the chin cup 305, but in other embodiments the neck strap 330 forms a complete loop, a portion of which is adhesively affixed to the surface of the chin cup 305.

FIG. 4 illustrates a perspective view of a releasable access tool 400 for use by disabled persons, featuring a spring-loaded neck retention element according to an embodiment of the present invention. The spring-loaded neck retention element includes a flexible spring-loaded curved element that is open at one side, has both a free end and an end that is affixed to a portion of a releasable access tool, and is adapted to be worn around the neck. The illustrated embodiment includes a chin cup 405, a chin cushion 410, a shaft 465, a shaft tip 470, and a spring-loaded neck retention element 480.

The chin cup 405 is affixed along its inner surface and along the outer circumference of its outer surface to the chin cushion 410. The spring-loaded neck retention element 480 is mechanically affixed to one side of the chin cup 405, with the free end of the spring-loaded neck retention element 408 remaining uncoupled to any other portion of the releasable access tool 400. The proximal end of the shaft 465 is adhesively affixed to the front surface of the chin cup 405, and the shaft tip 470 is mechanically coupled to the distal end of the shaft 465.

In operation, the releasable access tool 400 is placed around the neck of a disabled person (the user) by another person (an assistant). The assistant places the releasable access tool 400 around the neck of the user by grasping the free end of the spring-loaded neck retention element 480 and pulling the free end away from the releasable access tool 400. An assistant then places the spring-loaded neck retention element 480 around the neck of the user, with the chin cup 405 resting below the chin of the user on the user's lower neck or upper chest. To access the tool, the user lowers their chin to make contact between the chin and the inner concavity of the chin cup 405, and subsequently pushes the chin forward to the seat chin cup 405 and the chin cushion 410 on the front surface of the user's chin. The user then employs the muscles of the head and neck to lift the user's head. The spring-loaded neck retention element 480 maintains a backward pull on the chin cup 405, keeping the chin cup 405 and the chin cushion 410 affixed to the user's chin. The user moves the muscles of the face, head and neck to move the shaft 465 or the shaft tip 470 into physical contact with a target object. The user then manipulates the target object by applying force against the shaft 465. To disengage the tool, the user lowers the chin and presses the chin toward the neck and chest, allowing the tool to slip from the chin and rest in a neutral position around the neck of the user. An assistant removes the tool by grasping the free end of the spring-loaded neck retention element 480 and pulling the free end away from the releasable access tool 400. An assistant then lifts the spring-loaded neck retention element 480 away from the neck of the user.

In the illustrated embodiment, the spring-loaded neck retention element 480 is affixed to the chin cup 405. In other embodiments, the spring-loaded neck retention element 480 and the chin cup 405 are constructed as a single unit, with a chin cushion 410 that slips over the chin cup 405 to cover both the interior and exterior surfaces of the chin cup 405. In an embodiment, the chin cup 405 is affixed to the spring-loaded neck retention element 480 along two, three or four sides of the chin cup 405. In another embodiment, the chin cup 405 is affixed to the spring-loaded neck retention element 480 with a spring element. In some embodiments, the spring-loaded neck retention element 480 includes an elastomer, while in others, the spring-loaded neck retention element 480 includes a plastic. In an embodiment, the spring-loaded neck retention element includes a metal. In an embodiment, the surface of the spring-loaded neck retention element 480 that is placed against the neck of a user further includes a compressible material to cushion the neck of the user. Another embodiment includes a slidably adjustable cushion that can be moved along portions of the spring-loaded neck retention element 480. In some embodiments, the spring-loaded neck retention element 480 is shaped and sized to accommodate the neck size of a user. The spring-loaded neck retention element 480 is adapted in some embodiments to extend at its free end down some part of the body of a user such as the back, the chest or under the arm for added stability.

In some embodiments, the discontinuous curved neck retention element 480 is colorless and translucent. In other embodiments, the discontinuous curved neck retention element 480 is colorless and opaque. In an embodiment, the discontinuous curved neck retention element 480 is flesh-colored and translucent, while in another embodiment the discontinuous curved neck retention element 480 is flesh-colored and opaque. In an embodiment, the discontinuous curved neck retention element 480 has a color other than a flesh tone. In some embodiments, the discontinuous curved neck retention element 480 includes an electronic component.

In alternative embodiments, the spring-loaded neck retention element 480 is attached at its free end to an additional element that can be affixed to the chin cup 405 for added stability. In some embodiments this additional element is a hook, a snap, Velcro, a buckle or other mechanism. In other embodiments this additional element is an elastic strap that is affixed to the chin cup 405 by a hook, a snap, Velcro, a buckle, or other mechanism.

In an embodiment, the spring-loaded neck retention element 480 is rigid and is coupled to chin cup 405 with a spring-loaded element. In another embodiment, the spring-loaded neck retention element 480 is rigid and is coupled to the chin cup 405 with an elastic element, such as an elastic fabric, silicone or rubber.

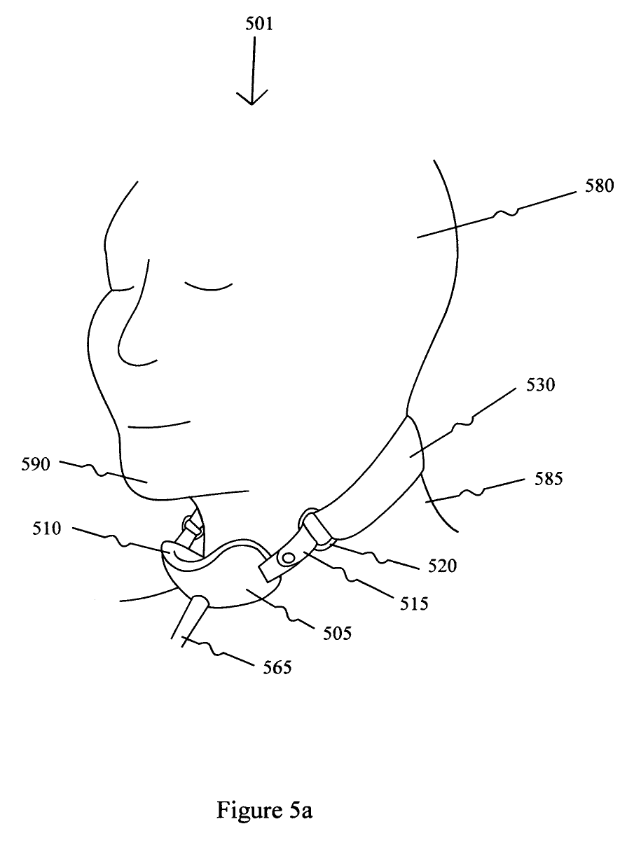

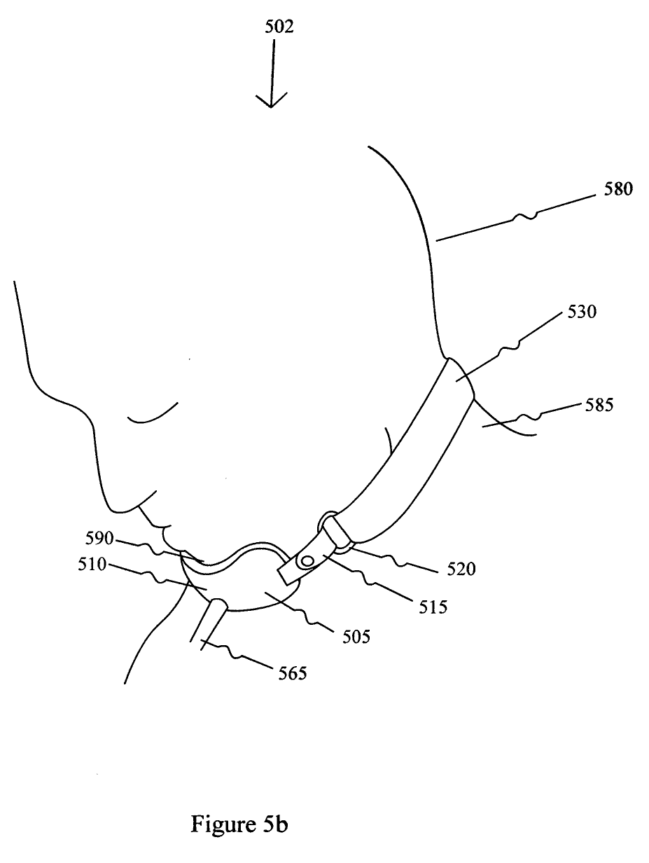

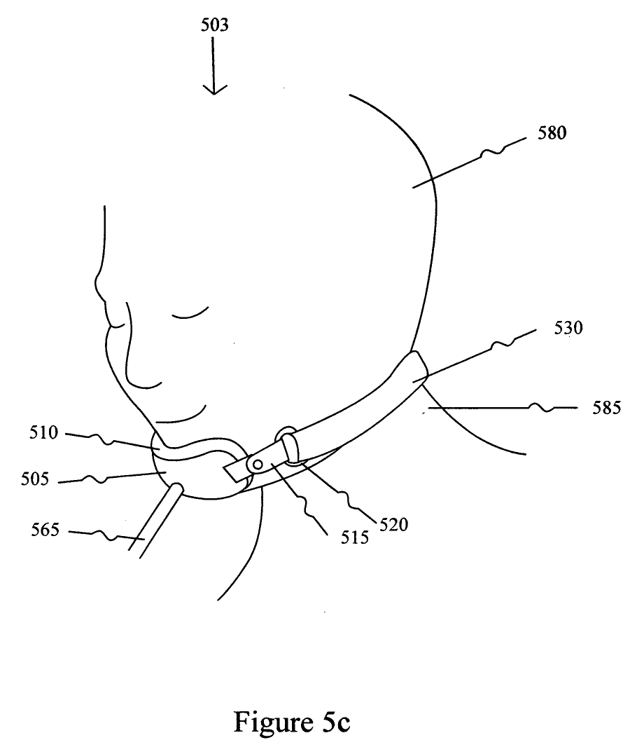

FIGS. 5a, 5b and 5c illustrate the positions of a releasable access tool 500 for use by disabled persons on a user engaging the tool in accordance with an embodiment of the present invention. FIG. 5a illustrates a first step 501 in the use of a releasable access tool according to an embodiment of the present invention, wherein a releasable access tool is suspended in a neutral position around the neck of a user prior to use. FIG. 5b illustrates a second step 502 in the use of a releasable access tool according to an embodiment of the present invention, wherein a user's head and neck are positioned to engage the releasable access tool. FIG. 5c illustrates a third step 503 in the use of a releasable access tool according to an embodiment of the present invention, wherein a user's head, chin and neck are positioned for use of the releasable access tool mounted upon the user's chin.

The illustrated embodiment includes a first step 501, a second step 502 and a third step 503. Each step further includes a chin cup 505 with an inner concavity, a chin cup cushion 510, a front strap 515, a ring connector 520, an elastic neck strap 530, a shaft 565, a user's head 580, a user's neck 585, and a user's chin 590.

In each of the first step 501, the second step 502 and the third step 503, the chin cup 505 is coupled to the front strap 515, which is coupled to a connector ring 520. The connector ring 520 is coupled to the elastic neck strap 530, which is coupled at its other end to another connector ring 520. This second connector ring 520 is coupled to the front strap 515. The front strap 515 is coupled to the chin cup 505, such that the chin cup 505, the front strap 515, the connector rings 520 and the elastic neck strap 530 form a continuous loop. The shaft 565 is coupled to the front surface of chin cup 505. The chin cup 505 is coupled along its inner surface and along the circumference of its outer surface to the chin cushion 110.

In the first step 501 of the illustrated embodiment, an assistant places the chin cup 505, the chin cushion 510, the front strap 515, the connector rings 520, the elastic neck strap 530 and the shaft 565 over a user's head 580 around a user's neck 585 such that the chin cup 505 is positioned on the lower neck or upper chest of the user below the user's chin 590 (see FIG. 5a). The elastic neck strap 530 is positioned around the back of the user's neck 585. The elastic neck strap 530 and the chin cup 505 are at rest around the user's neck 585 with the shaft 165 pointing downward along the user's chest until the user engages in the second step 502.

In the second step 502, the muscles of the user's head 580, the user's neck 585 and the user's chin 590 are used to pull the user's chin 590 down and inward toward the lower neck and chest of the user to place the user's chin 590 into contact with the interior concavity of the chin cup 505 and the chin cushion 510 (see FIG. 5b). The user need not see the chin cup 505 or the chin cushion 510 to accomplish this step. The elastic neck strap 530 remains positioned around the back of the user's neck 585, while the chin cup 505 and the chin cushion 510 cover some portion of the user's chin 590.

In the third step 503, the muscles of the user's head 580 and the user's neck 585 are used to push the user's chin 590 upward and outward from the lower neck and upper chest of the user in a scooping motion, stretching the elastic neck band 530 and seating the chin cup 505 and the chin cushion 510 securely over the front surface of the user's chin 590 (see FIG. 5c). The chin cup 505 and the chin cushion 510 are held in place on the front surface of the user's chin 590 by the rearward spring force provided by the elastic neck band 530. The position of the chin cup 505 on the front of the user's chin 590 holds the shaft 165 in a position projecting away from the user.

Some embodiments include a spring-loaded neck retention element instead of an elastic neck strap 530. In some embodiments, a spring-loaded neck retention element is coupled to one end of the chin cup 505 while the other end of the spring-loaded neck retention element is coupled to the user or is uncoupled to any other element. In some embodiments, the spring-loaded neck retention element is connected directly to the chin cup 505 while in other embodiments the spring-loaded neck retention element is coupled to the chin cup 505 with a strap, a spring, a ring or other element.

In other embodiments constructed without an elastic neck strap 530, one end of a spring-loaded retention element is coupled to the chin cup 505, while the other end of the spring-loaded retention element is coupled to a portion of a mobility device. For example, in one embodiment the other end of the elastic neck strap 530 is coupled to a pivoting arm connected to the head rest or neck rest of a motorized wheelchair. The chin cup 505 and the chin cushion 510 are held in position below the user's chin 590 by the pivoting arm and the spring-loaded retention element until the user engages the device.

In some embodiments that include a spring-loaded retention element coupled to a portion of a mobility device, the user grasps the chin cup or some element coupled to the chin cup with the user's mouth, teeth or tongue in order to move the chin cup into position for use below the user's chin. In an embodiment, the user then uses the muscles of the head, neck and face to pull the chin downward and inward toward the user's lower neck and upper chest, placing the user's chin into contact with the interior concavity of the chin cup or the chin cushion. The user subsequently engages the device by pushing the user's chin upward and outward away from the user's lower neck and chest. The spring-loaded retention element, which is coupled at one end to the mobility device, provides the rearward force to retain the chin cup against the chin of the user in these embodiments. The user later disengages the user's chin from the chin cup by using the muscles of the head, neck and face to pull the user's chin downward and inward toward the lower neck and chest, allowing the chin cup to drop from the chin. The chin cup remains in a resting position below the user's chin, where the user is able to reengage it as desired.

FIG. 6 illustrates a flow chart for a method of use of a releasable access tool according to an embodiment of the present invention. This embodiment includes a first step 601, a second step 602, a third step 603, and a fourth step 604.

In the first step 601 of the illustrated embodiment, an assistant positions an access tool around the neck of a user. In this embodiment, the access tool includes a chin cup with an inner concavity, a shaft coupled to the chin cup, and a rearward spring force element coupled to the chin cup. In some embodiments the rearward spring force element is an elastic neck strap. In other embodiments the rearward spring force element is a spring element that is placed around the neck. In some embodiments the spring force element is worn around the neck of the user, while in other embodiments the spring force element is coupled to a mobility device such as a motorized wheelchair. In an embodiment, an assistant positions the access tool around the neck of a user by stretching the spring element until the diameter of the access tool is larger than the head of the user and pulling the access tool downward over the head of the user until the access tool rests around the neck with the chin cup below the chin of the user. In other embodiments, an assistant positions the access tool around the neck of the user by uncoupling some portion of the rearward spring force element from another portion of the access tool, placing the access tool around the neck of the user, and subsequently coupling the rearward spring force element to some portion of the access tool. In an embodiment, the rearward spring force element is placed around the neck of the user by an assistant without uncoupling a free end of the rearward spring force element from any portion of the access tool.

In the second step 502, the user's chin is lowered toward the lower neck and chest of the user to place the user's chin into contact with the interior concavity of the chin cup. The user need not see the chin cup to accomplish this step. The rearward spring force element remains positioned around the back of the user's neck. In some embodiments, a chin cushion is coupled to the chin cup; in these embodiments the user's chin is placed into contact with the chin cushion during this step.

In the third step 503, the user's chin is pushed forward, stretching the rearward spring force element and placing tension on the chin cup. This step seats the chin cup on the chin of the user. In some embodiments, the rearward spring force element stretches along its length, while in other embodiments, the rearward spring force element does not stretch.

In the fourth step 504, the user's chin is raised upward and outward from the lower neck and upper chest of the user in a scooping motion, stretching the rearward spring force element and seating the chin cup securely over the front surface of the user's chin. The chin cup is held in place on the front surface of the user's chin by the rearward spring force provided by the rearward spring force element. The position of the chin cup on the front of the user's chin holds the shaft in a position projecting away from the user.

A number of head-mounted and mouth-held pointers have been described by others. While the prior art includes a variety of head pointers for use by persons lacking the use of hands or arms, one major disadvantage of these pointers has been that most must be mounted to the head of the user by an assistant prior to use and also removed by an assistant after use. While some users may have the ability to drop a mouth-held pointer when finished, the user is not able to re-engage the mouth-held pointer again without the assistance of another person. Furthermore, the mouth-held pointers cause the user to salivate and the use of such pointers may also cause jaw fatigue; the user is unable to speak, eat or drink while using the pointer, and breathing may also be compromised. Dental wear can become a problem for users of mouth-held pointers.

Many of the head-mounted prior art pointers are large and highly visible to others, causing social discomfort or embarrassment to the user. Such pointers also obscure the vision of the user when mounted to the forehead or to the top of the head, causing eyestrain and annoyance. While a small number of chin-mounted pointers have been described in the prior art, these pointers have been held to the user's chin by a strap or other fastener worn above the user's ears and around the back of the user's head. This design prevents the user from releasing the pointer and picking it up again without help from an assistant. Finally, many of the prior art pointers are prohibitively expensive to purchase and are uncomfortable to wear for long periods of time.

Consequently, one or more of the embodiments of the present invention provide an inexpensive, comfortable, versatile and visually unobtrusive chin-mounted pointer that is usable by those persons with limited or no use of their hands or arms. The pointer is coupled and uncoupled to the user's chin by the user without the assistance of others, hanging unobtrusively at rest around the lower neck of the user while not in use. When the user wishes to use the pointer, the user picks up the pointer by lowering the chin toward the chest and bringing it into contact with the chin cup. Pushing the chin forward and upward in a scooping motion seats the chin cup with pointer firmly on the user's chin, where it is used to physically manipulate objects such as keyboards and elevator buttons. The variety of pointer tips, any of which are easily added to the end of the pointer, increases the versatility of the device and allows the user to perform a number of different tasks without purchasing additional pointers. The placement of the strap behind the neck, the use of clear materials and the small size of the device make it less visually apparent to others, while the overall design ensures the comfort of the user. Users are able to eat, drink, speak, and breathe through the mouth while the pointer is in use. In addition, users are also able to operate a breath-activated sip/puff device such as a motorized wheelchair control while using the pointer.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. An access tool, including:
a rearward spring force element;
a chin cup formed with a concavity to accommodate a chin, wherein said chin cup is mechanically coupled to said rearward spring force element; and
a shaft with a first end and a second end, wherein said shaft is coupled at said first end to said chin cup.

2. The access tool of claim 1 wherein said rearward spring force element stretches along its length in response to applied force.

3. The access tool of claim 1 wherein said first end of said shaft is removably coupled to said chin cup.

4. The access tool of claim 2, wherein the length of said rearward spring force element is adjustable.

5. The access tool of claim 4 wherein said rearward spring force element further includes a releasable fastener.

6. The access tool of claim 1 further including a compressible element in mechanical contact with a surface of said chin cup.

7. The access tool of claim 1 wherein said chin cup further includes two apertures adapted to accommodate said shaft.

8. The access tool of claim 1 wherein said chin cup further includes a multiplicity of apertures adapted to accommodate said shaft.

9. The access tool of claim 1 wherein said rearward spring force element further includes a slidably adjustable cushion.

10. The access tool of claim 1 further including a tip component coupled to said second end of said shaft, wherein said tip component comprises a side wall, a closed bottom and an open top.

11. The access tool of claim 9 wherein a surface of said tip component is textured.

12. The access tool of claim 9 wherein said tip component accommodates a functional element.

13. The access tool of claim 1 wherein said shaft is hollow.

14. The access tool of claim 13 wherein said shaft is adapted to accommodate an electronic component.

15. The access tool of claim 1 wherein said shaft includes a curvature.

16. The access tool of claim 1 wherein said shaft is translucent and colorless.

17. The access tool of claim 1 wherein said chin cup is translucent and colorless.

18. The access tool of claim 6 wherein said flexible cushion is translucent and colorless.

19. The access tool of claim 1 wherein only one end of said rearward spring force element is coupled to said chin cup.

20. The access tool of claim 17 wherein said rearward spring force element is semi-rigid.

21. The access tool of claim 1 wherein one end of said rearward spring force element is coupled to said chin cup and wherein another end of said rearward spring force element is coupled to a mobility apparatus.

22. The access tool of claim 19 wherein said mobility apparatus is a motorized wheelchair.

23. The access tool of claim 19 wherein said another end of said rearward spring force element is movably coupled to said mobility apparatus and wherein said chin cup is positioned by a user's mouth.

24. The access tool of claim 1 wherein one end of said rearward spring force element is coupled to said chin cup and wherein another end of said rearward spring force element is coupled to a user.

25. A method for use of an access tool, including:
positioning an access tool circumferentially around a neck below a chin, wherein said access tool includes a chin cup formed with a concavity to accommodate said chin, an outward pointing shaft coupled to said chin cup and a rearward spring force element coupled to said chin cup;
lowering said chin to position said concavity against said chin;
pushing said chin forward, wherein said chin cup is biased against the chin by said rearward spring force element; and
raising said chin, wherein said chin cup remains biased against said chin by said rearward spring force element.

26. The method of claim 19 further including:
physically manipulating an object with said outward pointing shaft.

27. The method of claim 20 further including:
lowering said chin to disengage said chin from said chin cup.

28. The method of claim 18 further including:
Adding a tip component to said outward pointing shaft by placing one end of said shaft against said tip component and applying force along said outward pointing shaft, wherein said tip component comprises a side wall, a closed bottom and an open top.
